(19) United States
(12) Patent Application Publication
(10) Pub. No.: US 2025/0327102 A1
(43) Pub. Date: Oct. 23, 2025

(54) METHOD AND SYSTEM FOR RECOVERING OIL FROM A GRAIN DRY MILLING SYSTEM

(71) Applicant: Fluid Quip Technologies, LLC, Cedar Rapids, IA (US)

(72) Inventors: David Koziol, Fort Calhoun, NE (US); Christopher Kempf, Cedar Rapids, IA (US)

(21) Appl. No.: 19/184,333

(22) Filed: Apr. 21, 2025

Related U.S. Application Data
(60) Provisional application No. 63/637,049, filed on Apr. 22, 2024.

(51) Int. Cl. C12P 7/6436; C12P 7/6409

(52) U.S. Cl. CPC C12P 7/6436; C12P 7/6409

(57) ABSTRACT

An improved dry grind method and system for recovering oil from insoluble solids (fiber wet cake) separated from the whole stillage byproduct in a plant that derives a biochemical, such as alcohol (e.g., ethanol), from corn, wheat, or other cereal grains. In one embodiment, the method includes subjecting the fiber wet cake to one or more dryers to provide a desirable moisture content in a resulting dried fiber cake. Then, the dried fiber cake can be subjected to one or more oil press devices, such as an expeller press, that extracts bound oil from the dried fiber cake by squeezing or pressing the fiber cake under high pressure and at a desired temperature to provide an oil filtrate. The oil in the oil filtrate can be recovered via an oil recovery centrifuge and can be sold as a separate high value product.

### TECHNICAL FIELD

The present invention relates to dry grind methods and systems of alcohol production and recovering oil from a fiber wet cake separated from a whole stillage byproduct in a plant that derives a biochemical, such as alcohol (e.g., ethanol), from grain.

### BACKGROUND

Dry milling ethanol plants generally convert corn and/or other grains into typically only three products, i.e., ethanol, distillers grain oil, and distiller's grains with solubles. A typical grain dry milling process consists of four major steps: grain handling and milling, liquefaction and saccharification, fermentation and distillation, and co-product recovery. Grain handling and milling is the step in which the grain is brought into the plant and ground to promote better conversion of starch to glucose. Liquefaction is the step of converting solids, such as starch, to a flowable liquid producing oligosaccharides, and saccharification is where the oligosaccharides are converted into single glucose molecules. Fermentation is the process of yeast or bacteria, or as clostridia, for example, converting glucose into a biofuel or a biochemical/biomolecule, such as ethanol. Distillation is the process of removing the biofuel or biochemical/biomolecule, such as ethanol, from the fermentation product. Co-product recovery is the step in which the grain by-products are de-watered and made ready for market. There are many known chemical and biological conversion processes known in the art that utilize yeast, bacteria, or the like to convert glucose/sugar to other biofuels and biochemical/biomolecule components like ethanol, for example.

The recovery of alcohol, e.g., butanol, ethanol (a natural co-product), etc., and other similar compounds, generally begins with the beer (spent fermentation broth) being sent to a distillation system. With distillation, ethanol is typically separated from the rest of the beer through a set of stepwise vaporizations and condensations. The beer less the alcohol extracted through distillation is known as whole stillage, which contains a slurry of the spent grains including grain protein, fiber, oil, minerals, carbohydrates/sugars, and fermentation agent. This byproduct is too diluted to be of much value at this point and is further processed to provide the distiller's grains with solubles.

In typical processing, when the whole stillage leaves the distillation column, it is generally subjected at the back end of the process to a decanter centrifuge to separate insoluble solids or “wet cake”, which includes mostly fiber as well as germ that includes bound oil, from the liquid or “thin stillage”, which includes, e.g., protein, fine fiber, free oil, and amino acids. After separation, the thin stillage moves to evaporators to boil away moisture, leaving a thick syrup that contains the soluble (dissolved) solids. The concentrated syrup is typically mixed with the wet cake, and the mixture may be sold to beef and dairy feedlots as distillers wet grain with solubles (DWGS). Alternatively, the wet cake and concentrated syrup mixture may be dried in a drying process and sold as distillers dried grain with solubles (DDGS). The resulting DDGS generally has a crude protein content of about 29% and a fat (oil) content of 5% and is a useful feed for cattle and other ruminants mainly due to its protein and fiber content.

While DDGS and DWGS provide a critical secondary revenue stream, for example, that offsets a portion of the overall ethanol production cost, it would be beneficial to provide a method and system where oil, which can be sold as a separate high value product, can be additionally obtained from the fiber wet cake separated from the whole stillage byproduct thereby increasing oil yield in the method and system.

### SUMMARY OF THE INVENTION

The present invention is directed to a method and system for recovering oil from a whole stillage byproduct in a plant that derives a biochemical, such as alcohol (e.g., ethanol), from a dry milling of grains including, for example, corn and wheat. The oil, which can be sold as a separate high value product, can be obtained from the insoluble solids (wet cake) separated from the whole stillage byproduct to increase oil yield in the method and system. This oil recovery can be in addition to other oil recovery in the method and system to increase overall oil yield.

In one embodiment, a method for recovering oil from an insoluble solids portion is provided that includes separating a whole stillage byproduct into an insoluble solids portion, including fiber and germ with bound oil, and a solubles portion, including soluble solid, and drying the insoluble solids portion to provide a dried insoluble solids portion, including the fiber and germ. The dried insoluble solids portion then is pressed to free bound oil from the germ and to provide a first filtrate, including the free oil, and a first deoiled insoluble solids portion, including pressed fiber and germ, and oil from the first filtrate is recovered to provide an oil co-product.

In another embodiment, a method for recovering oil from an insoluble solids portion is provided that includes conditioning distillers grains with or without solubles that defines an insoluble solids portion and includes fiber and germ with bound oil by mixing and/or rehydrating the distillers grains with or without solubles. Then, the conditioned distillers grains with or without solubles is pressed to free bound oil from the germ and to provide a first filtrate, including the free oil, and a first deoiled distillers grains with or without solubles, including pressed fiber and germ, and oil is recovered from the first filtrate to provide an oil co-product.

In another embodiment, a system for recovering oil from an insoluble solids portion is provided that includes a first apparatus that receives the whole stillage byproduct and is configured to separate the whole stillage byproduct into an insoluble solids portion, including fiber and germ with bound oil, and a solubles portion, including soluble. A first drying device is situated after the first apparatus and receives the insoluble solids portion. The first drying device dries the insoluble solids portion to provide a dried insoluble solids portion, including the fiber and germ. A second apparatus is situated after the first drying device and receives the dried insoluble solids portion. The second apparatus is configured to press the dried insoluble solids portion to free bound oil from the germ and provide a first filtrate, including the free oil, and a first deoiled insoluble solids portion, including pressed fiber and germ. An oil recovery device is situated after the second apparatus and receives the first filtrate. The oil recovery device is configured to recover oil from the first filtrate to provide an oil co-product.

### BRIEF DESCRIPTION OF THE DRAWINGS

The accompanying drawings, which are incorporated in and constitute a part of this specification illustrate embodiments of the invention and, together with the general description of the invention given above and the detailed description given below, serve to explain the principles of the invention. Similar reference numerals are used to indicate similar features throughout the various figures of the drawings.

FIG. 1 is a flow diagram of a typical dry grind alcohol production process;

FIG. 2 is a flow diagram of a method and system for recovering oil in a dry grind biochemical process in accordance with an embodiment of the invention;

FIG. 2A is a variation of the flow diagram of FIG. 2 that shows a method and system for recovering oil in a dry grind biochemical process in accordance with another embodiment of the invention;

FIG. 2B is a variation of the flow diagram of FIG. 2 that shows a method and system for recovering oil in a dry grind biochemical process in accordance with another embodiment of the invention;

FIG. 3 is a flow diagram of a method and system for recovering oil in a dry grind biochemical process in accordance with another embodiment of the invention;

FIG. 3A is a variation of the flow diagram of FIG. 3 that shows a method and system for recovering oil in a dry grind biochemical process in accordance with another embodiment of the invention;

FIG. 3B is a variation of the flow diagram of FIG. 3 that shows a method and system for recovering oil in a dry grind biochemical process in accordance with another embodiment of the invention;

FIG. 4 is a flow diagram of a method and system for recovering oil in a dry grind biochemical process in accordance with another embodiment of the invention;

FIG. 4A is a variation of the flow diagram of FIG. 4 that shows a method and system for recovering oil in a dry grind biochemical process in accordance with another embodiment of the invention; and

FIG. 4B is a variation of the flow diagram of FIG. 4 that shows a method and system for recovering oil in a dry grind biochemical process in accordance with another embodiment of the invention.

### DETAILED DESCRIPTION OF SPECIFIC EMBODIMENTS

The present invention relates to dry grind methods and systems of alcohol and/or biochemical production for recovering oil from whole stillage in a plant that derives alcohol (e.g., ethanol) from corn, wheat, or other cereal grains. The oil, which can be sold as a separate high value product, can be obtained from the wet (fiber) cake separated from the whole stillage byproduct and can be in addition to other oil recovery in the method and system to increase overall oil yield.

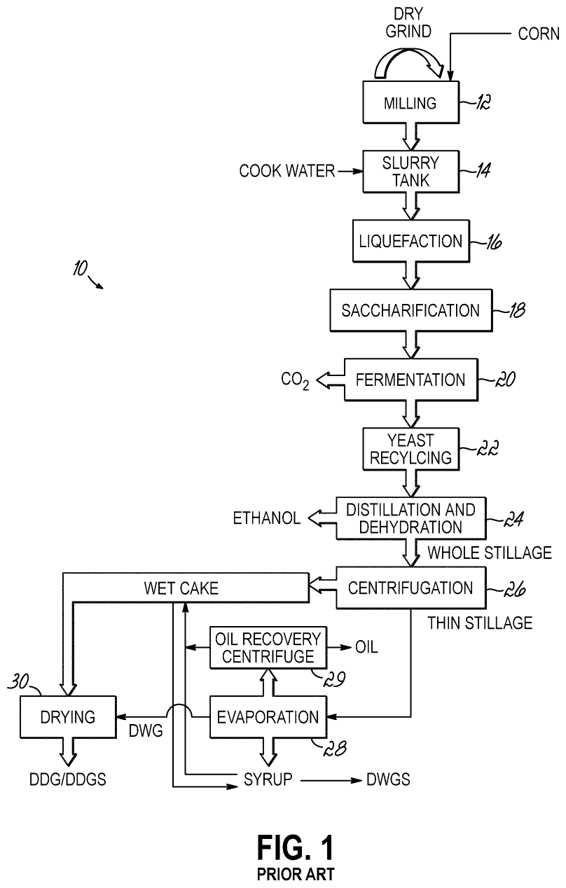

FIG. 1 shows a flow diagram of a typical dry grind alcohol (e.g., ethanol) production method 10. Although virtually any type and quality of grain, such as but not limited to sorghum, wheat, triticale, barley, rye, tapioca, cassava, potato, pea and other starch and/or oil containing grains and/or legumes can be used to produce ethanol and/or a biochemical/biomolecule, for example, the feedstock for this process is typically corn referred to as “No. 2 Yellow Dent Corn.” Also, as a general reference point, the dry grind method 10 can be divided into a front end and a back end. The part of the method 10 that occurs prior to distillation and dehydration 24 is considered the “front end,” and the part of the method 10 that occurs after distillation and dehydration 24 is considered the “back end.” To that end, the front end of the dry grind method 10 begins with a milling step 12 in which dried whole corn kernels can be passed through hammer mills for grinding/milling into meal or a fine powder. The screen openings in the hammer mills or similar devices typically are of a size 6/64 to 9/64 inch, or about 2.38 mm to 3.57 mm, but some plants can operate at less than or greater than these screen sizes. The resulting particle distribution yields a very widely spread, bell type curve, which includes particle sizes as small as 45 microns and as large as 2 mm to 3 mm. The majority of the particles tend to be in the range of 500 to 1200 microns, which is the “peak” of the bell curve. Other screen openings larger and smaller can be deployed in the hammer mills. Other milling devices such as roller mills or pin mills can also be utilized in the dry grain grinding step.

After the milling step 12, the ground meal is mixed with cook water to create a slurry at the slurry tank 14 and a commercial enzyme called alpha-amylase is typically added (not shown). Creating the slurry at the slurry tank 14 is followed by a liquefaction step 16 whereat the pH can be adjusted to about 4.8 to 5.8 and the temperature maintained between about 50°° C. to 105° C. so as to convert the insoluble starch in the slurry to soluble starch. The stream after the liquefaction step 16 has about 30% dry solids (DS) content, but can range from about 29-36%, with all the components contained in the corn kernels, including starch/sugars, protein, fiber, starch, germ, grit, oil, and salts, for example. Higher solids are achievable, but this requires extensive alpha amylase enzyme to rapidly breakdown the viscosity in the initial liquefaction step. There generally are several types of solids in the liquefaction stream: fiber, germ, and grit.

Liquefaction 16 may be followed by separate saccharification and fermentation steps, 18 and 20, respectively, although in most commercial dry grind ethanol processes, saccharification and fermentation can occur simultaneously. This single step is referred to in the industry as “Simultaneous Saccharification and Fermentation” (SSF). Both saccharification and

SSF can take as long as about 50 to 60 hours. Gluco-Amylase enzyme is typically added to the fermentation step 20 that facilitates the further breakdown of the starches and larger polysaccharides into single monomer sugar molecules that the yeast consumes to produce ethanol (or other similar alcohols) and carbon dioxide. Yeast can optionally be recycled in a yeast recycling step 22 either during the fermentation process or at the very end of the fermentation process. Yeast produced during the fermentation process will pass through to the distillation and dehydration step 24. In addition to the gluco-amylase being added other enzymes can be added (such as but not limited to phytase, protease, cellulase, hemicellulose, xylanase, beta-glucanase, and the like) that can further enhance protein and oil recovery downstream. Subsequent to the fermentation step 20 is the distillation and dehydration step 24, which utilizes a still to recover the alcohol (e.g., ethanol).

Finally, a centrifugation step 26 involves centrifuging the residuals, i.e., “whole stillage”, which includes the non-fermentable grain components (protein, free oil, fiber, ash, and minerals, for example) and yeast yielded from the distillation and dehydration step 24 in order to separate the insoluble solids (“wet cake”) from the liquid (“thin stillage”). The liquid from the centrifuge contains about 5% to 12% DS. The “wet cake” includes fiber, of which there generally are three types: (1) pericarp, with average particle sizes typically about 1 mm to 3 mm; (2) tipcap, with average particle sizes about 500 micron; (3) and fine fiber, with average particle sizes of about 250 microns. There may also be proteins and yeast bodies with a particle size of about 45 microns to about 300 microns as well as germ including bound oil. The fiber and other fractions may contain bound protein that is chemically and or physically attached to the fiber and other fraction.

The thin stillage typically enters evaporators in an evaporation step 28 in order to boil or flash away moisture, leaving a thick syrup which contains the soluble (dissolved) solids (mainly protein and starches/sugars) from the fermentation (25 to 40% dry solids) along with residual oil and fine fiber. The concentrated slurry can be sent to a centrifuge to separate the oil from the syrup in an oil recovery step 29. The oil can be sold as a separate high value product. The oil yield is normally about 0.8 lb/bu to 1.0 lb/bu of corn with elevated free fatty acids content compared to traditional wet mill corn oil. This oil yield recovers only about ⅓ to ½ of the oil in the corn, with part of the oil passing with the syrup stream and the remainder being lost with the fiber/wet cake stream. About one-half of the oil inside the corn kernel remains inside the germ and fiber fraction after the distillation and dehydration step 24, which cannot be separated in the typical dry grind process using centrifuges as the oil is bound, not free. The free fatty acids content, which is created when the oil is heated and exposed to oxygen throughout the front and back-end process, reduces the value of the oil. The (de-oil) centrifuge only removes less than 50% because the protein and oil make an emulsion, which cannot be satisfactorily separated without the use of chemicals or added mechanical separation unit operations.

The syrup, which has more than 10% oil, can be mixed with the centrifuged wet cake, and the mixture may be sold to beef and dairy feedlots as Distillers Wet Grain with Solubles (DWGS). Alternatively, the wet cake and concentrated syrup mixture may be dried in a drying step 30 and sold as Distillers Dried Grain with Solubles (DDGS) to dairy and beef feedlots. Optionally, the wet cake (fiber fraction) may be dried in drying step 30 and sold as Distillers Dried Grains, where the syrup from step 28 is not added to the wet cake stream and similarly sold as Distillers Dried Grain (DDG) to dairy and beef feedlots. The DDGS has all the corn and yeast protein and about 67% of the oil in the starting corn material. But the value of DDGS is low due to the high percentage of fiber, and in some cases the oil is a hindrance to animal digestion and lactating cow milk quality. As such, it would be beneficial to remove oil, which can be sold as a separate high value product, upstream of the DDGS, such as from the insoluble solids (wet cake) stream.

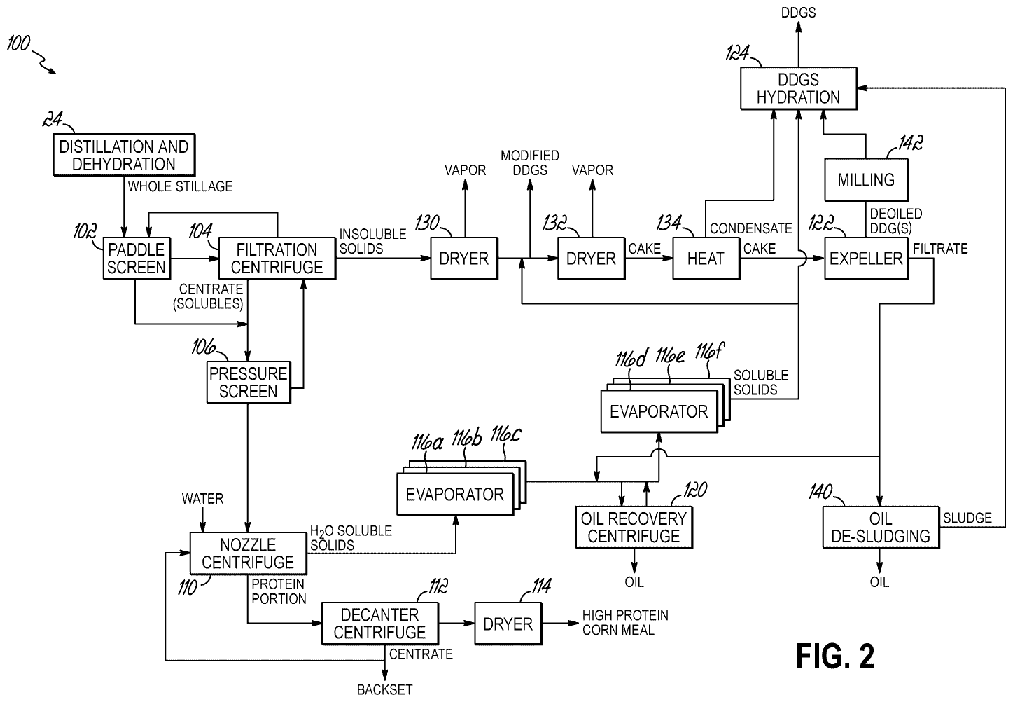

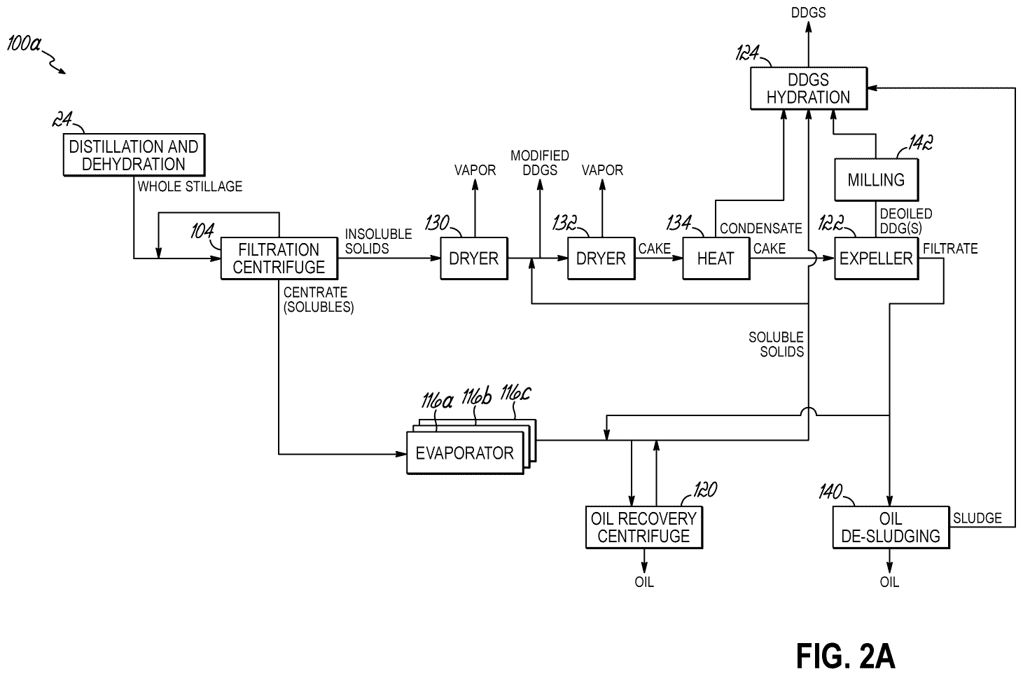

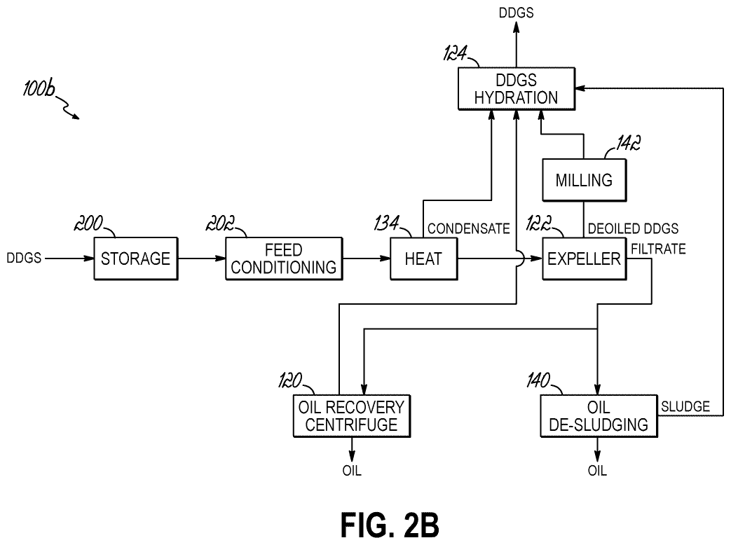

In accordance with the present invention, FIG. 2 shows one embodiment of a method and system for recovering oil in a dry grind process, collectively numeral 100, from a whole stillage byproduct such as produced in a typical corn dry-milling method and system 10 like that just described in FIG. 1. While a typical whole stillage byproduct is utilized here, it should be understood that the whole stillage from any corn or similar or other grain dry milling process may be utilized with the same or similar results. Again, the whole stillage byproduct contains a slurry of soluble and insoluble solids, i.e., spent yeast and spent grains from the distillation and dehydration step 24, which can include amino acids, protein, fiber, and oil (free and bound), for example, that can be processed in accordance with embodiments of this invention to recover oil therefrom, which can be sold as a separate high value product.

With reference to FIG. 2, the whole stillage is piped from the dry milling distillation and dehydration step 24 and may be subjected to one or more paddle screens 102 with openings sized to permit the passage of protein particles and yeast cells but reject/prevent the passage of most fiber particles and germ (with bound oil). The material that passes through the paddle screen(s) 102 is higher in protein due, for example, to the exclusion of fiber by the paddle screens. The optional paddle screen 102 or other like equipment, as discussed below, can be situated before a centrifuge 104, such as a filtration centrifuge, which also is further discussed below, so as to aid ultimately in separation of the insoluble solids portion, e.g., fiber, from the centrate (solubles) portion by initially filtering out desirable amounts of water, amino acids, protein, free oil, and, incidentally, small fiber fines from the whole stillage byproduct. This initial screening can help reduce the resulting load on the subsequent centrifuge 104. The resulting throughs (centrate) from the paddle screen 102 eventually joins with the centrate (solubles) underflow from the centrifuge 104, as will be discussed in greater detail below.

To filter the whole stillage byproduct, the optional paddle screen 102 (or like equipment) can include screen openings of no greater than about 400 microns. In another example, the paddle screen 102 can include openings therein of no greater than about 250 microns. In yet another example, the openings therein are no greater than about 150 microns. In yet another example, the openings therein are no greater than about 100 microns. In yet another example, the openings therein are no greater than about 75 microns. It should be understood that these values are exemplary and that those of ordinary skill in the art will recognize how to determine the size of the openings to achieve the desired separation. In one example, the optional paddle screen 102 is a standard type paddle screen as is known in the art. One such suitable paddle screen 102 is the FQ-PS32 available from Fluid-Quip, Inc. of Springfield, Ohio. It should be understood that the optional paddle screen 102 may be replaced with other types of filtration/separation or pre-concentration devices, e.g., a standard pressure screen, conic centrifuge, cyclone, filter press, rotary filter, or hydroclone, which can perform the desired filtration or preconcentration function. One such suitable pressure screen is the PS-Triple available from Fluid-Quip, Inc. of Springfield, Ohio. In addition, although a single paddle screen 102 is depicted, it should be understood that a plurality of paddle screens may be situated in-line, either in series or in parallel, and utilized for filtering the whole stillage byproduct.

The whole stillage from the distillation and dehydration step 24, if the optional paddle screen 102 (or like equipment) is not present, or the cake (solids) from the optional paddle screen 102 is sent to the centrifuge 104, such as a filtration centrifuge, whereat the whole stillage byproduct or overflow is separated into the insoluble solids portion (wet cake), which includes fiber and germ (with bound oil), and the centrate (solubles) portion, which includes amino acids, protein, oil, etc. One such suitable filtration centrifuge 104 is described in Lee et al., U.S. Pat. No. 8,813,973 entitled “Apparatus and Method for Filtering a Material from a Liquid Medium”, the contents of which are expressly incorporated by reference herein in its entirety. The centrifuge 104 may be configured to perform both the initial filtering (sometimes referred to as a pre-concentration) of the whole stillage byproduct and washing of the fiber so as to clean the fiber and remove the protein, amino acids, free oil, and other components that remain associated with the fiber after the initial filtration or pre-concentration.

With respect to the centrifuge 104, such as a filtration centrifuge, the washing of the fiber may include a washing cycle, wherein the fiber is mixed and rinsed with a liquid or in wash water, followed by a de-watering cycle, wherein the wash water is separated from the fiber. The washing of the fiber may include multiple rinsing/de-watering cycles (in series or parallel). Additionally, a counter current washing technique may be employed to reduce wash water usage through the system. After washing the fiber, but before the fiber exits the centrifuge, the fiber may go through an enhanced de-watering stage, a compaction stage, and/or an air drying stage to further de-water or dry the fiber. This may increase the dryer capacity or eliminate the dryer altogether. Eventually, the washed and filtered fiber exits the centrifuge 104 so that the fiber can be further processed, as discussed further below to ultimately result in a desired product, such as DWG(S) or DDG(S), and to recover oil still bound within the wet (fiber) cake. In one example, at least a portion of the fiber wet cake may be transported to a remote site for further processing, such as anaerobic or aerobic digestion, conversion to C5 and C6 sugar molecules for biofuel, or biochemical conversion or food production processes. Moreover, any separated out portion of slurry from the fiber wet cake, e.g., protein, free oil, amino acids, water/wash water, etc., which occurs via screening, is collected to define the centrate (solubles) stream, then transported and further processed as described below. Optionally, a portion of the slurry and/or wash water may be piped back to the optional paddle screen 102 for further reprocessing. The centrifuge 104 may provide the filtered material at a water concentration of between about 55% and about 85% water, which is a significant reduction compared to conventional filtration systems.

With continuing reference to FIG. 2, although a single centrifuge 104 is depicted, it should be understood that a plurality of centrifuges, either in parallel or series, may be situated in-line and utilized for separating the whole stillage byproduct into its insoluble solids portion (wet fiber cake) and centrate (solubles) portion. In an alternate embodiment, it is contemplated that the centrifuge 104 can be or be replaced by a standard pressure screen, decanter centrifuge (e.g,, a solid bowl decanter), paddle screen, desludging device, dewatering press, or other like devices as are known in the art to separate the whole stillage byproduct into the insoluble solids portion (wet cake) and centrate (solubles) portion. One such suitable pressure screen is the PS-Triple available from Fluid-Quip, Inc. of Springfield, Ohio. One such suitable decanter centrifuge is the NX-944HS available from Alfa Laval of Lund, Sweden. And one such suitable paddle screen is the FQ-PS32 available from Fluid-Quip, Inc. of Springfield, Ohio.

As further shown in FIG. 2, the centrate (solubles) underflow from the centrifuge 104 is piped to join up with the centrate from the optional paddle screen 102 prior to or at an optional standard pressure screen 106, as is known in the art, to further optionally aid in separation of any fine fiber from the centrate (solubles) portion. If the optional paddle screen 102 is not present, the centrate (solubles) underflow from the centrifuge 104 is sent to the optional pressure screen 106. Prior to being subjected to the pressure screen 106, the protein content within this stream ranges from 17% to 34% and solids content within this stream ranges from 5% to 15%.

Fine fiber having a particle size less than that of the screen of the centrifuge 104 and/or optional paddle screen 102 may pass through and to subsequent steps of the corn dry-milling process. At the pressure screen 106, the fine fiber can be separated from the centrate (solubles) and piped back to the centrifuge 104 whereat the fine fiber may be filtered out. To separate the fine fiber, in one example, the pressure screen 106 can include screen openings of no greater than about 500 microns. In another example, the pressure screen 106 can include openings therein no greater than about 400. In another example, the pressure screen 106 can include openings therein no greater than about 250. In another example, the pressure screen 106 can include openings therein no greater than about 150 microns. In yet another example, the pressure screen 106 can include openings therein of no greater than about 75 microns. One such suitable pressure screen 106 is the PS-Triple available from Fluid-Quip, Inc. of Springfield, Ohio. In an alternate embodiment, the pressure screen 106 may be replaced with a standard paddle screen or decanter centrifuge, as are mentioned above, or other like device or particle size separation operation, to aid in separation of the fine fiber from the centrate (solubles) portion. In addition, although a single pressure screen 106 is depicted, it should be understood that a plurality of pressure screens, either in parallel or series, may be situated in-line and utilized for filtering the centrate (solubles) underflow.

After the optional pressure screen 106, the centrate (solubles) underflow is then piped and subjected to one or more disc nozzle centrifuges 110, which can be arranged in series or parallel. The nozzle centrifuge 110 can be provided with washing capabilities so that water, or similar aqueous solutions or low solid centrate streams, along with the centrate (solubles) portion, can be supplied to the nozzle centrifuge 110. The additional water/liquid allows for easier separation of the centrate (solubles) into a protein portion and a water soluble solids portion. In these nozzle centrifuges 110, the denser material including the protein particles can be concentrated in a high density underflow stream, which is referred to as a protein portion. The lighter material including most of the free oil and some of the lighter (fine) fiber material passes to a lower density overflow stream. One such suitable nozzle centrifuge 110 is the FQC-950 available from Fluid-Quip, Inc. of Springfield, Ohio. In an alternate embodiment, the nozzle centrifuge 110 can be replaced with a standard cyclone apparatus or other like device, as are known in the art, to separate the centrate (solubles) portion into the underflow protein portion and overflow water soluble solids portion. One such suitable cyclone apparatus is the RM-12-688 available from Fluid-Quip, Inc. of Springfield, Ohio.

The underflow stream from the disc nozzle centrifuge 110, which defines the protein portion, can be collected in a tank (not shown). Water can be added to the tank to dilute the underflow stream and help in washing soluble non protein solids from the high protein portion/slurry increasing the purity of a final protein meal. The underflow stream can be separated into a liquid fraction and a protein wet cake fraction at one or more decanting centrifuges (e.g., a decanter centrifuge) 112. That is, at the decanter centrifuge 112, the protein portion is dewatered to provide a dewatered protein portion (protein wet cake). The decanter centrifuge 112 is standard and known in the art. One such suitable decanter centrifuge 112 is the SG806 available from Alfa Laval of Lund, Sweden. In addition, although a single decanter centrifuge 112 is depicted, it should be understood that a plurality of decanter centrifuges may be situated in-line, either in series or parallel, and utilized for filtering the centrate (solubles) underflow. In an alternate embodiment, the decanter centrifuge 112 may be replaced with a standard filter press or rotary vacuum, or other like device, as are known in the art, to dewater the centrate (solubles) portion. A water portion or centrate from the decanter centrifuge 112 may be recycled back, for example, as backset to the slurry tank 14, the liquefaction step 16, or the fermentation step 20 for reuse in the dry-milling process. In another example, the centrate from the decanter centrifuge 112 may be recycled back to one or more of the optional paddle screen 102 and/or the optional pressure screen 106. In addition, at least a portion of the centrate may be returned back to the nozzle centrifuge 110, as is shown here, such as for use as wash water.

The dewatered protein portion or protein wet cake, which contains most of the protein, from the decanter centrifuge 112 can be further optionally dried, such as by being sent to a dryer 114, e.g., a rotary dryer, a rotary steam tube dryer, spray dryer, a ring dryer, a crystallizer, or an air classifier, as is known in the art, to make a high protein meal product. In another embodiment, the dewatered protein portion can be subjected to vacuum filtration or other drying methods, or other downstream processes prior to or after being dried, as are known in the art. The final dried protein product defines a high protein corn meal that includes, for example, at least 40 wt % protein on a dry basis and which may be sold as swine feed, chicken feed, aqua feed, food uses, or have other uses, including pharmaceutical and/or chemical usage, for example. In another embodiment, the high protein corn meal includes at least 45 wt % protein on a dry basis. In another embodiment, the high protein corn meal includes at least 50 wt % protein on a dry basis. In yet another embodiment, the high protein corn meal includes at least 60 wt % protein on a dry basis. In still another embodiment, the high protein corn meal includes about 56 wt % protein on a dry basis. It should be understood that the type and concentration of the protein present in the final product may vary based on the carbohydrate-containing grain source, the fermentation process, and/or the specific application. The resulting high protein corn meal may be sold at a much higher price per ton than DDGS or DWGS.

Returning now to the overflow stream or separated water soluble solids portion, which includes free oil as well as minerals and soluble proteins, the overflow stream optionally can be sent to a set of three evaporators 116a, 116b, and 116c, as are known in the art, whereat water/liquid is removed to begin to thicken the water soluble solids and oil stream to a high solids syrup. Thereafter, all or a portion of the water soluble solids portion can be piped and subjected to an optional oil recovery centrifuge 120 so that oil can be removed therefrom. A 2 or a 3-phase oil recovery centrifuge may be utilized here. One such suitable oil recovery centrifuge is the ORPX 617 available from Alfa Laval of Lund, Sweden. In one example, the recovered oil product here can include between about 40 wt % to about 60 wt % of the total corn oil in the corn. The remainder of the water soluble solids portion from the evaporators 116a-c and/or from the optional oil recovery centrifuge 120 can be piped and optionally subjected to another set of three evaporators 116d, 116e, and 116f whereat water/liquid portion is further evaporated from the water soluble solids portion to ultimately yield a soluble solids portion (or syrup). While the water soluble solids portion is shown subjected to two sets of three evaporators 116a-c, 116d-f, it should be understood that the number of evaporators and sets thereof can be varied, i.e., can be more or less, from that shown depending on the particular application and result desired.

At least a portion of the resulting soluble solids portion may be combined with a deoiled insoluble solids portion received from an oil press device 122, such as an expeller press or the like, as discussed in greater detail below, to provide a distillers dry grains with solubles (DDGS), which may be further hydrated at hydration step 124 and/or sold to dairy and beef feedlots. In addition, as shown in FIG. 2, optionally at least a portion of the soluble solids stream can be combined with the insoluble solids/wet cake stream just downstream of the centrifuge 104, such as after a first dryer 130, as discussed in greater detail below.

In another example, at least a portion of the soluble solids portion (syrup) may be directly recovered and used as a natural fertilizer or as a feed source for an aerobic or and anaerobic digestion process. In another example, at least a portion of the soluble solids portion may be directly recovered for use as a raw material feed source for conversion to simple sugar, which then can be further converted to biofuel or used in other biochemical processes, for example. Additionally, at least a portion of the soluble solids stream can be directly recovered and further processed as a raw material feed source, such as for a bio-digester to produce biofuels and/or biochemicals, an algae feed source, and/or further processed via fermentation, for example, to yield a high protein nutrient feed. Accordingly, in such a dry-milling process, neither the DDG nor DWG would receive the typical concentrated syrup from the evaporators 116a-f. Yet, despite the potential lower protein content, the DDG and DWG may still be sold to beef and dairy feedlots as cattle feed or other animal feed markets.

Further concerning FIG. 2 and returning to the wet (fiber) cake or insoluble solids portion that is separated from the liquid stream at the centrifuge 104, the wet cake, which includes primarily fiber along with germ including bound oil, can be subjected to the first dryer 130, e.g., rotary dryer, a rotary steam tube dryer, a spray dryer, a ring dryer, a crystallizer, or the like, of a desired temperature to remove moisture therefrom in the form of water vapor so as to provide a desired moisture level in a dried fiber cake. The moisture content of the dried fiber cake after the first dryer 130, for example, can be about 40% or more. In another example, the moisture content of the dried fiber cake can be about 30% or more. The temperature of the first dryer 130 can be from about 175° F. to about 250° F. In another example, the temperature can be from about 205° F. to about 225° F. In another example, the temperature can be from about 205° F. to about 215° F. In another example, the temperature of the first dryer 130 can be about 210° F. These are exemplary examples of moisture and temperature and one skilled in the art can determine the optimal conditions as needed for oil recovery.

As indicated above and in one example, a portion of the soluble solids portion (or syrup) from the set of three evaporators 116d-f can be combined with the dried fiber cake here, i.e., after the first dryer 130, to provide a modified distillers dry grains with solubles (MDGS), which can include dry matter content in a range of about 45% to 50% by weight of the MDGS. More specifically, the soluble solids portion may be combined with the dried fiber cake after the first dryer 130 and before an optional second dryer 132. After combining the soluble solids with the dried fiber cake after the first dryer 130, a portion of the modified DGS may be separated from the stream, as shown in FIG. 2, and can be sold to beef and dairy feedlots as cattle feed or other animal feed markets, for example.

Next, all or a portion of the initially dried fiber cake from the first dryer 130, with or without the solubles/syrup, can be subjected to the second dryer 132, e.g., e.g., a rotary dryer, a rotary steam tube dryer, a spray dryer, a ring dryer, a crystallizer, or the like, of a desired temperature to continue to remove moisture therefrom in the form of water vapor so as to provide a desired moisture level in the dried fiber cake. The moisture content of the dried fiber cake after the second dryer 132, for example, can be from about 2% to 12%. In another example, the moisture content of the dried fiber can be from about 2% to 9%. In another example, the moisture content of the dried fiber can be from about 1% to 5% or from about 2% to 4%. In another example, the moisture content of the dried fiber cake after the second dryer 132 can be about 2%. In another example, the moisture content of the dried fiber cake can be from about 5% or less. In another example, the moisture content of the dried fiber cake can be from about 4.5% or less. In another example, the moisture content of the dried fiber cake after the second dryer 132 can be from about 4% or less. The temperature of the second dryer 132 can be from about 175° F. to about 250° F. In another example, the temperature of can be from about 205° F. to about 225° F. In another example, the temperature can be from about 205° F. to about 215° F. In another example, the temperature of the second dryer 132 can be about 210° F. The temperature of the second dryer 132 may be the same as, lower, or higher than the temperature of the first dryer 130, as desired. These are exemplary examples of moisture and temperature and one skilled in the art can determine the optimal conditions as needed for oil recovery.

The further dried fiber cake from the optional second dryer 132, with or without the solubles/syrup, can be subjected to an optional and additional heating step(s) 134 whereat the further dried fiber cake can be subjected to a heated device, such as a heated screw conveyer (e.g., a steam jacketed screw conveyer), or the like, and/or a third dryer, such as a trim dryer or the like, at a desired temperature(s) to remove any additional or desired moisture from the fiber cake in the form of water vapor/condensate so as to provide a desired moisture level in the further dried fiber cake. The moisture content of the further dried fiber cake after the optional and additional heating step(s) 134, for example, can be from about 2% to 9%. In another example, the moisture content of the dried fiber cake can be from about 1% to 5% or from about 2% to 4%. In another example, the moisture content of the dried fiber cake can be about 2%. In another example, the moisture content of the dried fiber cake can be from about 5% or less. In another example, the moisture content of the dried fiber cake can be from about 4.5% or less. In another example, the moisture content of the dried fiber cake after the optional and additional heating step(s) 134 can be from about 4% or less. The temperature of the heated device(s), such as the screw conveyor and/or third dryer, of the optional and additional heating step(s) 134 can be from about 175° F. to about 250° F. In another example, the temperature can be from about 205° F. to about 225° F. In another example, the temperature can be from about 205° F. to about 215° F. In another example, the temperature can be about 210° F. The temperature of the heated devices of the optional and additional heating step(s) 134 may be the same as, lower, or higher than the temperature of one another, the first dryer 130, or the second dryer 132, as desired.

In an alternative embodiment the first and/or second dryers 130, 132 may be replaced by a press, such as a screw press, or the like to produce or provide the same or similar results/resulting dried fiber cake as with use of the first and second dryers 130, 132. The screw press may be heated. One such suitable screw press is understood to be the Cone J2500 available from Conveyor Engineering Company of Cedar Rapids, Iowa. With the use of a screw press, for example, the moisture content of the resulting pressed/dried fiber cake initially may be higher than when the first and/or second dryers 130, 132 are used to dry the fiber cake. In one example, the moisture content of the pressed/dried fiber cake after the screw press, for example, can be about 55% or more. In another example, the moisture content of the dried fiber cake can be about 50% or more. In another example, the moisture content of the dried fiber cake can be about 45% or more. In another example, the moisture content of the dried fiber cake after the screw press can be about 40% or more. The temperature of the heated screw press can be from about 175° F. to about 250° F. In another example, the temperature can be from about 205° F. to about 225° F. In another example, the temperature can be from about 205° F. to about 215° F. In another example, the temperature of the heated screw press can be about 210° F. These are exemplary examples of moisture and temperature and one skilled in the art can determine the optimal conditions as needed for oil recovery.

The dried fiber cake from the second dryer 132 if the optional and additional heating step(s) 134 are not present or the dried fiber cake from the optional and additional heating step(s) 134 next can be subjected to the heated oil press device 122, such as an expeller press (or oil press), which can be a screw type machine that extracts bound oil from the dried fiber cake by squeezing or pressing the fiber cake under high pressure, such as through a caged barrel-like cavity, and at a desired temperature. In one example, the oil press device 122 uses friction and continuous pressure from the screw drive to move and compress the fiber cake. The end result is that bound oil is pressed out of the fiber cake and exits the oil press device 122 via desirably sized openings that prevents or limits the fiber cake from exiting therethrough with the oil. One such suitable oil press device is understood to be the Super Duo™ 55 available from Anderson International Corporation of Stow, Ohio.

The moisture content of the pressed fiber cake after the oil press device 122 can be from about 2% to 9%. In another example, the moisture content of the pressed fiber cake can be from about 1% to 5% or from about 2% to 4%. In another example, the moisture content of the pressed fiber cake can be about 2%. In another example, the moisture content of the pressed fiber cake can be from about 5% or less. In another example, the moisture content of the pressed fiber cake can be from about 4.5% or less. In another example, the moisture content of the pressed fiber cake after the oil press device 122 can be from about 4% or less. The temperature of the oil press device 122 can be from about 175° F. to about 250° F. In another example, the temperature can be from about 205° F. to about 225° F. In another example, the temperature can be from about 205° F. to about 215° F. In another example, the temperature of the oil press device 122 can be about 210° F. The temperature of the oil press device 122 may be the same as, lower, or higher than the temperature of the first dryer 130, the second dryer 132, or the heated device of the additional heating steps 134, as desired. In one example, the pressure of the oil press device 122 can be from 1 psi to about 6,000 psi. In another example, the pressure can be from about 3,000 psi to about 5,000 psi. In another example, the pressure can be from about 1,000 psi to about 3,000 psi. In another example, the pressure can be from about 2,000 psi to about 4,000psi. In still another example, the pressure can be greater than 6,000 psi. These are exemplary examples of moisture, pressure, and temperature and one skilled in the art can determine the optimal conditions as needed for oil recovery.

In an alternate embodiment, it is contemplated that the oil press device 122, such as the expeller press, can be replaced by a standard screw press. Although a single oil press device 122 is depicted, it should be understood that a plurality of oil press devices, e.g., expeller presses, either in parallel or series, may be situated in-line and utilized for extracting oil from the dried fiber cake by squeezing the fiber cake under high pressure. In one example, a first expeller press can be followed by a second expeller press in which the resulting deoiled insoluble solids can be combined together and the oil filtrate combined together as well and/or treated as discussed hereinabove and below.

The pressed or expelled oil filtrate from the oil press device 122 can be sent to an oil desludging step 140 whereat the oil filtrate can be subjected to a second oil recovery centrifuge so that oil can be separated from sludge and removed therefrom. A 2 or a 3-phase oil recovery centrifuge may be utilized here. One such suitable oil recovery centrifuge is the ORPX 617 available from Alfa Laval of Lund, Sweden. In another example, the oil recovery centrifuge could be replaced with a decanter or a settling tank that uses gravity to settle the sludge and purge. In one example, the recovered oil product here can include from about 1 wt % to about 25 wt % of the total corn oil in the corn. In another example, the recovered oil product can include from about 10 wt % to about 20 wt % of the total corn oil in the corn. In another example, the recovered oil product can include from about 10 wt % to about 15 wt % of the total corn oil in the corn. In still another example, the recovered oil product can include from about 15 wt % to about 25 wt % of the total corn oil in the corn. The resulting sludge (also sometimes referred to as foots) can include a mixture of residual oil and fines, which can include protein and fine fiber, that may be combined with the deoiled DDG(S) and soluble solids, which can be further hydrated at hydration step 124. Alternatively, all or a portion of the oil filtrate can be sent to the oil recovery centrifuge at step 120, such as by being rejoined with the water soluble solids portion just after the first set of evaporators 116a-c, as shown in FIG. 2.

All or a portion of the deoiled insoluble solids (which may or may not include soluble solids, as indicated above) from the oil press device 122 can be combined with soluble solids from the optional second set of evaporators 116d-e and/or condensate from the optional and additional heating step(s) 134. In one example and prior to combining the same with the soluble solids, condensate, and sludge, the deoiled insoluble solids may be subjected to a milling step 142, which can include a hammer mill, roller mill, pin mill, or the like, to break up the insoluble solids and provide a desirable average particle size. Alternatively, the deoiled insoluble solids may be subjected to a screw mixer, such as a twin screw mixer and the like. In addition, although a single milling device is noted above, it should be understood that a plurality of milling devices, either in parallel or series, may be situated in-line and utilized for breaking up the insoluble solids and providing a desirable average particle size. In one example, a portion of the deoiled DDGS prior to dehydration step 124 (and before or after optional milling step 142) may be separated out and sold to beef and dairy feedlots as cattle feed or other animal feed markets. In one example, the moisture content of the deoiled DDGS/fiber cake can be about 2% or greater here.

As indicated above, condensate from the optional and additional heating step(s) 134 can be combined with the combined soluble solids portion and deoiled insoluble solids portion, along with the sludge, at step 124 to further hydrate/rehydrate the combination to provide a distillers dry grains with solubles (DDGS), which may be sold to dairy and beef feedlots. In one example, the soluble solids portion and deoiled insoluble solids portion (whether optionally subjected to the milling step 142 or not) and sludge can be combined in paddle mixer or the like and the condensate misted over the combined materials to provide a desired hydration thereof at hydration step 124. The resulting DDGS can be hydrated to include a moisture content of from about 2% to 9%. In another example, the moisture content can be from about 3 to 9% or from about 4% to 9%. In another example, the moisture content of the resulting DDGS can be about 9%. In one example, the moisture content can be about 2% or greater. In another example, the moisture content can be about 3% or greater. In another example, the moisture content can be about 4% or greater. In another example, the moisture content of the resulting DDGS can be about 9% or greater. These are exemplary examples of final moisture and one skilled in the art can determine the optimal conditions as needed for oil recovery.

The resulting DDGS may be sold to beef and dairy feedlots as cattle feed or other animal feed markets, such as fish feed or the pet food market. In one example, the DDGS may be food grade and fit for human consumption and optionally be combined with yeast, for example.

In accordance with the present invention, FIG. 2A shows a variation of the method and system of FIG. 2 for recovering oil in a dry grind biochemical process. Here, in this method and system for recovering oil in a dry grind process, collectively numeral 100a, the optional paddle screen 102, optional pressure screen 106, and optional second set of evaporators 116d-f in the method 100 of FIG. 2 have been removed. In addition, the nozzle centrifuge 110 of FIG. 2, likewise, is considered optional here and has been removed such that the centrate (solubles)/thin stillage from the centrifuge 104 can be sent directly to evaporators 116a-c, and not the water soluble solids portion. In one example and as discussed above with respect to FIG. 2, the centrifuge 104 can be replaced with a decanter centrifuge, such as a solid bowl decanter. Notably, in view of the removal of the nozzle centrifuge 110, the protein portion is not separated out of the centrate and a high protein corn meal is not produced in this method 100a. Instead, the protein remains with the centrate, which is subjected to evaporation at evaporators 116a-c and further processed in accordance with the detailed discussion of the method 100 of FIG. 2 to increase oil yield/recovery yet provide for a DDGS that includes a higher protein content relative to the DDGS of FIG. 2, which may be sold to beef and dairy feedlots as cattle feed or other animal feed markets, such as fish feed or the pet food market, for example.

In accordance with the present invention, FIG. 2B shows another variation of the method and system of FIG. 2 for recovering oil in a dry grind biochemical process. Here, in this method and system for recovering oil a dry grind biochemical process, collectively numeral 100b, it has been adapted to receive DDGS, for example, that has been produced via a dry grind biochemical process at a different source/location, e.g., an alcohol plant, which could employ a method and system that is the same as or similar to that shown and described in FIG. 1, 2, or 2A. The received DDGS initially may be stored at storage step 200 and optionally conditioned at feed conditioning step 202 before being subjected to further treatment at additional heat step(s) 134, as described above in FIG. 2, to recover oil still contained therein. At optional feed conditioning step 202, the DDGS can be subjected to a mixing device, such as a paddle mixer or the like, to break up DDGS chunks, for example, and help provide a generally uniform particle size therein. Liquid, e.g., water, can be added to the mixing device, as needed/desired, to adjust the moisture content of the DDGS. The temperature of the DDGS may be adjusted, as needed/desired, such as by heating the same before, at, and or after the mixing device, which can also assist in moisture control of the DDGS. In addition, the DDGS can be treated with a processing aid such as a binding agent, e.g., starch, steam, syrup, and the like, that can be added to the DDGS, such as before, at, and/or after the mixing device, to allow for fines (such as fine fiber) to be bound with the larger pericarp fiber particles and pass through the oil press device 122, allowing for greater oil recovery and less downstream processing needs to remove the fines at the oil de-sludging step 140. Other processing aids alternately or additionally may be used as well including, for example, enzymes, such as phytase, protease, cellulase, hemicellulase, xylanase, beta-glucanase, and the like, flocculants, surfactants, demulsifiers, pH adjusters, such as sulfuric acid, hydrochloric acid, sodium hydroxide, and the like, which can further assist with oil recovery.

With continuing reference to FIG. 2B, the DDGS, which may be optionally conditioned, now can be subjected directly to additional heating step(s) 134 and further processed in accordance with the detailed discussion of the method 100 of FIG. 2 to increase oil yield/recovery. It is noted that while both the oil recovery centrifuge 120 and the oil desludging step 140 are shown in FIG. 2B, only one of these may be present/needed. That is, all of the oil filtrate from the expeller/oil press device 122 can be sent to the oil recovery centrifuge 120 or to the oil de-sludging step 140. And, as shown, all other steps/machinery for processing whole stillage have been removed. As such, with this standalone DDGS/oil recovery method and system 100b, there is no high protein grain (corn) meal produced with this specific setup, as with the method and system 100 of FIG. 2. In addition, if used, there is no soluble solids stream being sent from the oil recovery centrifuge 120 to the hydration step 124, for example, but rather the remaining filtrate from the oil filtrate stream is sent, which would be akin to the sludge from the oil de-sludging step 140. Also, while DDGS is shown here as having been received from another location, it should be understood that MDGS or DWGS may be contemplated as options for use in this method and system 100b as well.

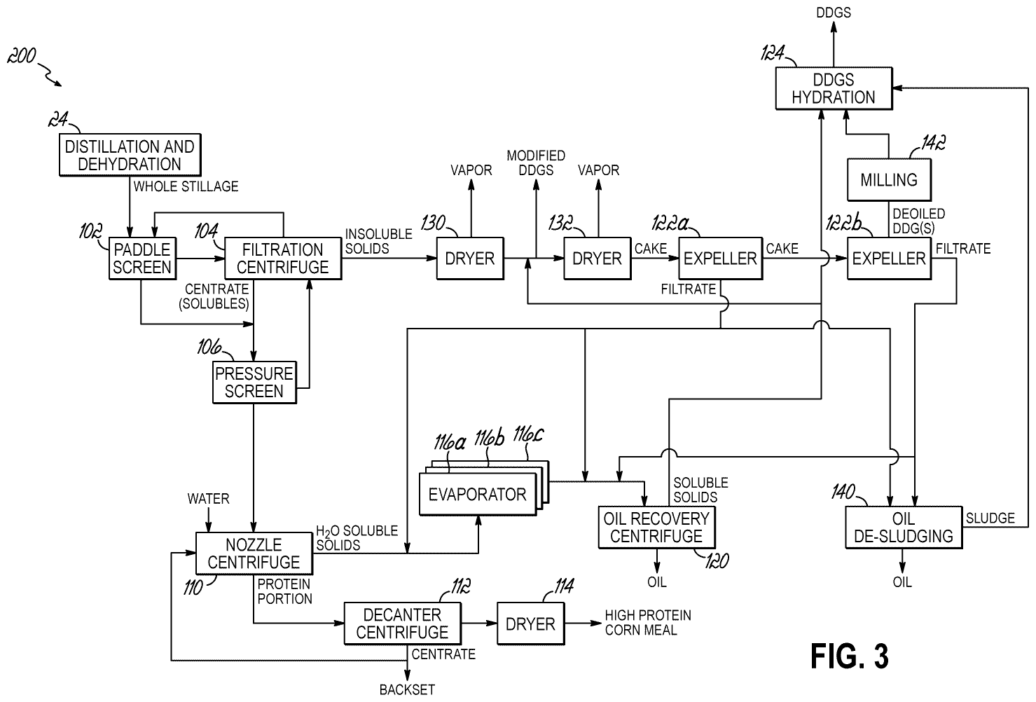

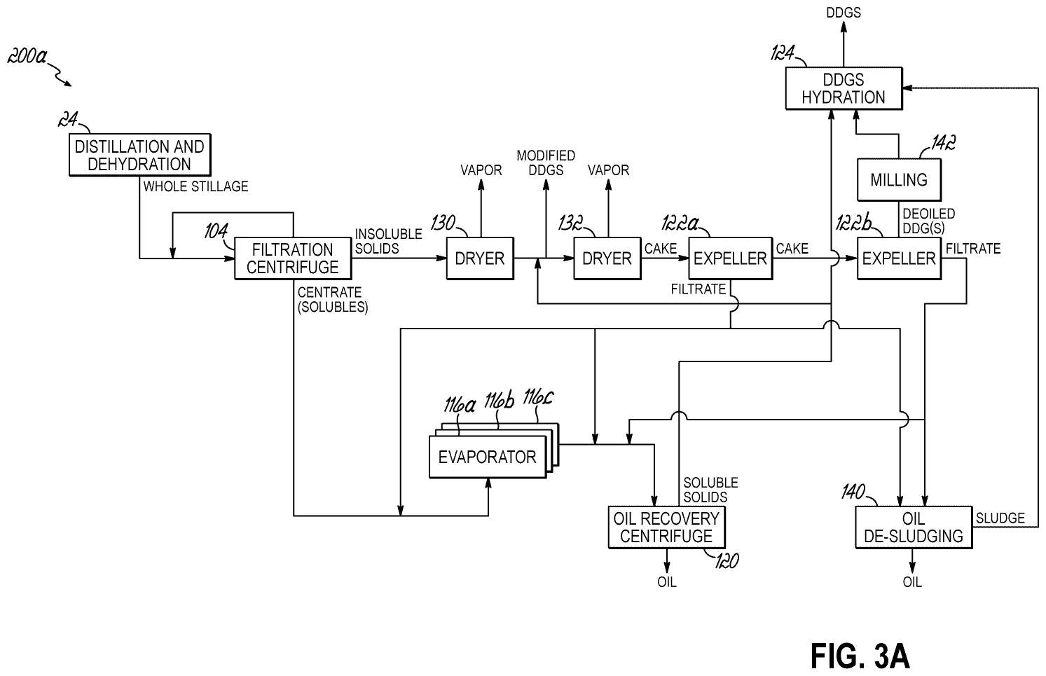

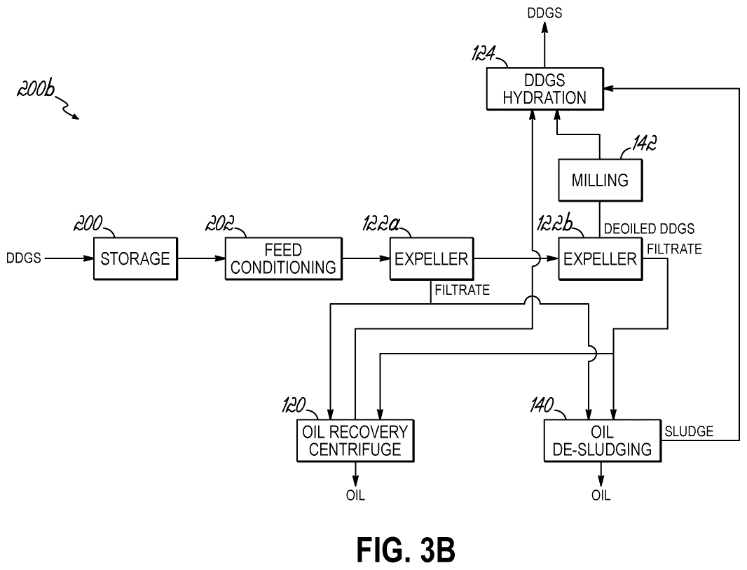

In accordance with the present invention, FIG. 3 shows another embodiment of a method and system for recovering oil in a dry grind process, collectively numeral 200, like the method and system just described in FIG. 2 but with variations thereto. To that end, certain reference numerals used in FIG. 2 are used here to represent like devices and/or steps in the method and system 100. The variations of the embodiment of FIG. 3 are discussed hereinbelow.

As shown in FIG. 3, the optional and additional heating step(s) 134 of FIG. 2 have been removed such that the further dried fiber cake from the second dryer 132 is sent and subjected directly to a first oil press device 122a, e.g., a first expeller press, to extract oil from the dried fiber cake by pressing the fiber cake to recover oil trapped within the fiber cake. The pressed fiber cake then can be sent and subjected to a second oil press device 122b, e.g., a second expeller press, to again extract additional oil from the pressed fiber cake by further pressing the pressed fiber cake to recover additional oil trapped within the fiber cake. As indicated above, it is contemplated that the expeller press can be replaced by a standard screw press. All or a portion of the further pressed fiber cake or deoiled insoluble solids (which may or may not include soluble solids, as indicated above) from the second oil press device can be combined with soluble solids and further subjected to hydration step 124 including initially being subjected to milling step 142 prior to the hydration step 124, as discussed above.

In one example, the moisture content of the pressed fiber cake after the first and second oil press devices 122a, 122b can be from about 2% to 9%. In another example, the moisture content of the pressed fiber cake can be from about 1% to 5% or from about 2% to 4%. In another example, the moisture content of the pressed fiber cake can be about 2%. In another example, the moisture content of the pressed fiber cake can be from about 5% or less. In another example, the moisture content of the pressed fiber cake can be from about 4.5% or less. In another example, the moisture content of the pressed fiber cake can be from about 4% or less. The temperature of the first and second oil press devices 122a, 122b can be from about 175° F. to about 250° F. In another example, the temperature can be from about 205° F. to about 225° F. In another example, the temperature can be from about 205° F. to about 215° F. In another example, the temperature can be about 210° F. The temperature of the first and second oil press devices 122a, 122b may be the same as, lower, or higher than the temperature of one another, the first dryer 130, or the second dryer 134, as desired. In one example, the pressure used in the first and second oil press devices 122a, 122b can be from 1 psi to about 6,000 psi. In another example, the pressure can be from about 3,000 psi to about 5,000 psi. In another example, the pressure can be from about 1,000 psi to about 3,000 psi. In another example, the pressure can be from about 2,000 psi to about 4,000 psi. In still another example, the pressure can be greater than 6,000 psi. The pressure of the first and second oil press devices 122a, 122b may be the same as, lower, or higher than the pressure of one another. These are exemplary examples of moisture, pressure, and temperature and one skilled in the art can determine the optimal conditions as needed for oil recovery.

The oil filtrate from the first oil press device 122a can be sent to oil recovery centrifuge 120, such as by being rejoined with the water soluble solids portion just after the evaporators 116a-c and/or combined with the water soluble solid portion prior the evaporators 116a-c, as shown in FIG. 3. The oil filtrate from the second oil press device 122b can be sent to the oil desludging step 140 whereat the oil filtrate can be subjected to the second oil recovery centrifuge so that oil can be removed therefrom. All or a portion of the oil filtrate from the first oil press device 122a also can be sent to the oil desludging step 140. The resulting sludge may be combined with the deoiled DDG(S) and soluble solids, which can be further hydrated, as discussed above. Alternatively, all or a portion of the oil filtrate from the second oil press 122b, like the oil filtrate from the first oil press 122a, can be sent to oil recovery centrifuge 120, such as by being rejoined with the water soluble solids portion just after the evaporators 116a-c, as shown in FIG. 3.

Also, in FIG. 3, the optional second set of evaporators 116d-e has been removed such that at least a portion of the soluble solids from the oil recovery centrifuge 120 may be combined directly with the deoiled insoluble solids portion received from the second oil press device 122b to provide a distillers dry grains with solubles (DDGS), which may be further hydrated and/or sold to dairy and beef feedlots. In addition, as shown in FIG. 3, at least a portion of the soluble solids stream from the oil recovery centrifuge 120 can be combined with the insoluble solids stream just downstream of the centrifuge 104, such as after the first dryer 130, as discussed above.

In accordance with the present invention, FIG. 3A shows a variation of the method and system of FIG. 3 for recovering oil in a dry grind biochemical process. Here, in this method and system for recovering oil in a dry grind process, collectively numeral 200a, the optional paddle screen 102 and the optional pressure screen 106 in the method 200 of FIG. 3 have been removed. In addition, the nozzle centrifuge 110 of FIG. 3, likewise, is considered optional here and has been removed such that the centrate (solubles)/thin stillage from the centrifuge 104 can be sent directly to evaporators 116a-c, and not the water soluble solids portion. In one example and as discussed above with respect to FIG. 3, the centrifuge 104 can be replaced with a decanter centrifuge, such as a solid bowl decanter. Notably, in view of the removal of the nozzle centrifuge 110, the protein portion is not separated out of the centrate and a high protein corn meal is not produced in this method 200a. Instead, the protein remains with the centrate, which is subjected to evaporation at evaporators 116a-c and further processed in accordance with the detailed discussion of the method 200 of FIG. 3 to increase oil yield/recovery yet provide for a DDGS that includes a higher protein content relative to the DDGS of FIG. 3, which may be sold to beef and dairy feedlots as cattle feed or other animal feed markets, such as fish feed or the pet food market, for example.

In accordance with the present invention, FIG. 3B shows another variation of the method and system of FIG. 3 for recovering oil in a dry grind biochemical process. Here, in this method and system for recovering oil a dry grind biochemical process, collectively numeral 200b, it has been adapted, similar to FIG. 2B, to receive DDGS, for example, that has been produced via a dry grind biochemical process at a different source/location, e.g., an alcohol plant, which could employ a method and system that is the same as or similar to that shown and described in FIG. 1, 3, or 3A. The received DDGS initially may be stored at storage step 200 and optionally conditioned at feed conditioning step 202 before being subjected to further treatment at first expeller/first oil press device 122a, as described above in FIG. 3, to recover oil still contained therein. At optional feed conditioning step 202, the DDGS can be subjected to a mixing device, such as a paddle mixer or the like, to break up DDGS chunks, for example, and help provide a generally uniform particle size therein. Liquid, e.g., water, can be added to the mixing device, as needed/desired, to adjust the moisture content of the DDGS. The temperature of the DDGS may be adjusted, as needed/desired, such as by heating the same before, at, and or after the mixing device, which can also assist in moisture control of the DDGS. In addition, the DDGS can be treated with a processing aid such as a binding agent, e.g., starch, steam, syrup, and the like, that can be added to the DDGS, such as before, at, and/or after the mixing device, to allow for fines (such as fine fiber) to be bound with the larger pericarp fiber particles and pass through the oil press device 122, allowing for greater oil recovery and less downstream processing needs to remove the fines at the oil de-sludging step 140. Other processing aids alternately or additionally may be used as well including, for example, enzymes, such as phytase, protease, cellulase, hemicellulase, xylanase, beta-glucanase, and the like, flocculants, surfactants, demulsifiers, pH adjusters, such as sulfuric acid, hydrochloric acid, sodium hydroxide, and the like, which can further assist with oil recovery.

With continuing reference to FIG. 3B, the DDGS, which may be optionally conditioned, now can be subjected directly to first oil press device 122a and further processed in accordance with the detailed discussion of the method 200 of FIG. 3 to increase oil yield/recovery. And, as shown, all other steps/machinery for processing whole stillage have been removed. As such, with this standalone DDGS/oil recovery method and system 200b, there is no high protein grain (corn) meal produced with this specific setup, as with the method and system 200 of FIG. 3. In addition, it is noted that while both the oil recovery centrifuge 120 and the oil desludging step 140 are shown in FIG. 3B, only one of these may be present/needed. That is, all of the oil filtrate from both the first oil press device 122a and the second oil press device 122b can be sent to the oil recovery centrifuge 120 or to the oil de-sludging step 140. And, if used, there is no soluble solids stream being sent from the oil recovery centrifuge 120 to the hydration step 124, for example, but rather the remaining filtrate from one or both of the oil filtrate streams is sent, which would be akin to the sludge from the oil de-sludging step 140. Also, while DDGS is shown here as having been received from another location, it should be understood that MDGS or DWGS may be contemplated as options for use in this method and system 200b as well.

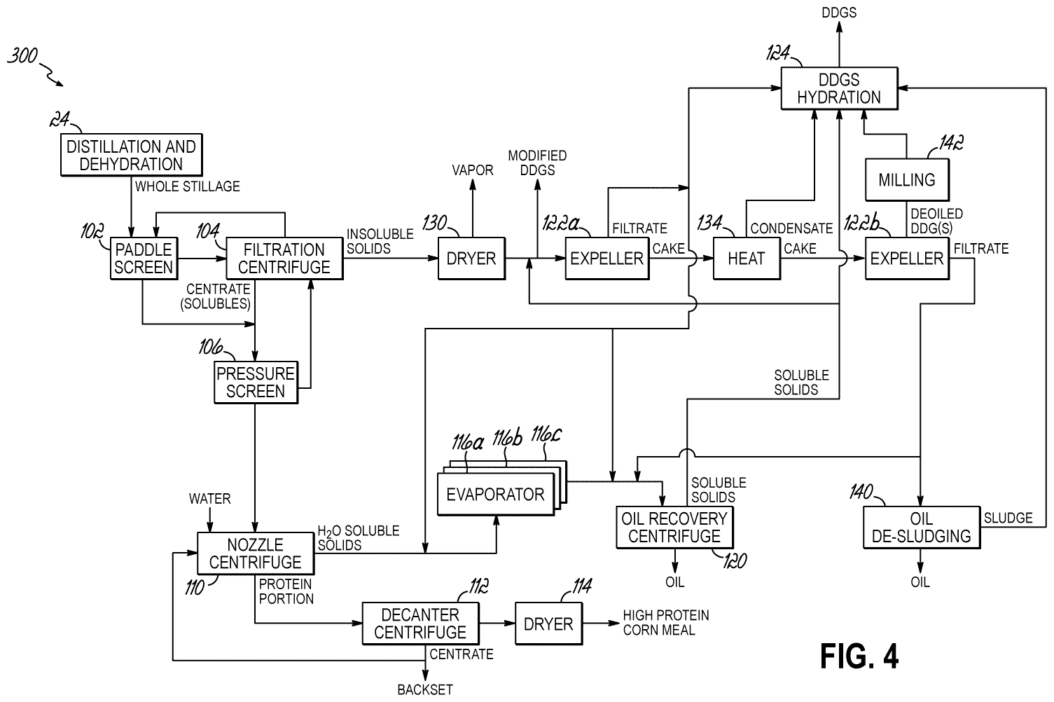

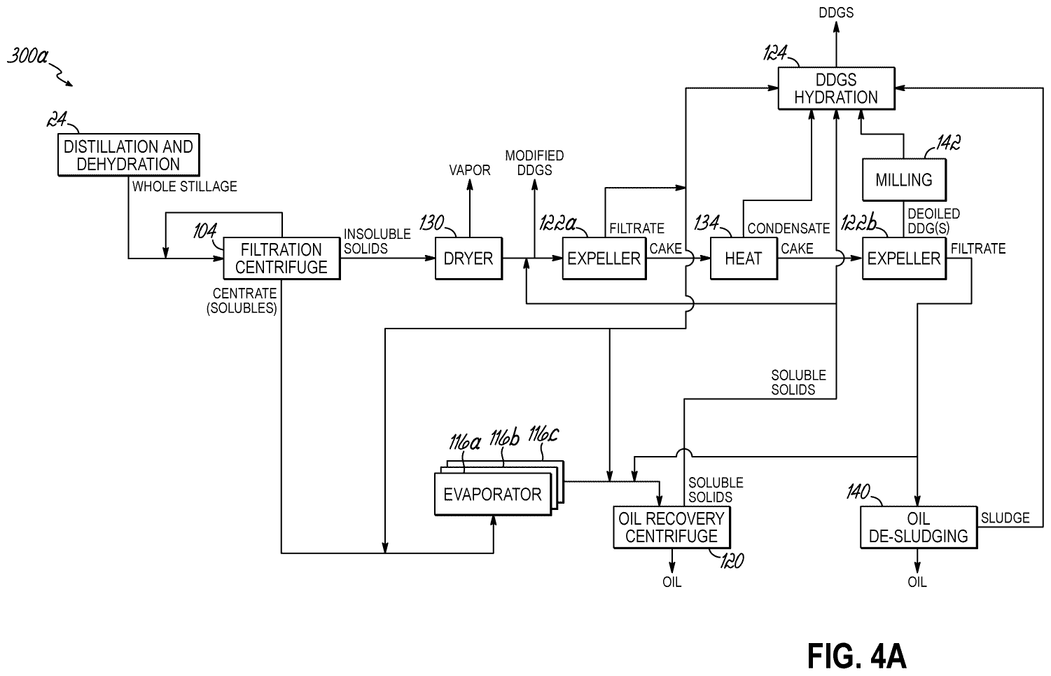

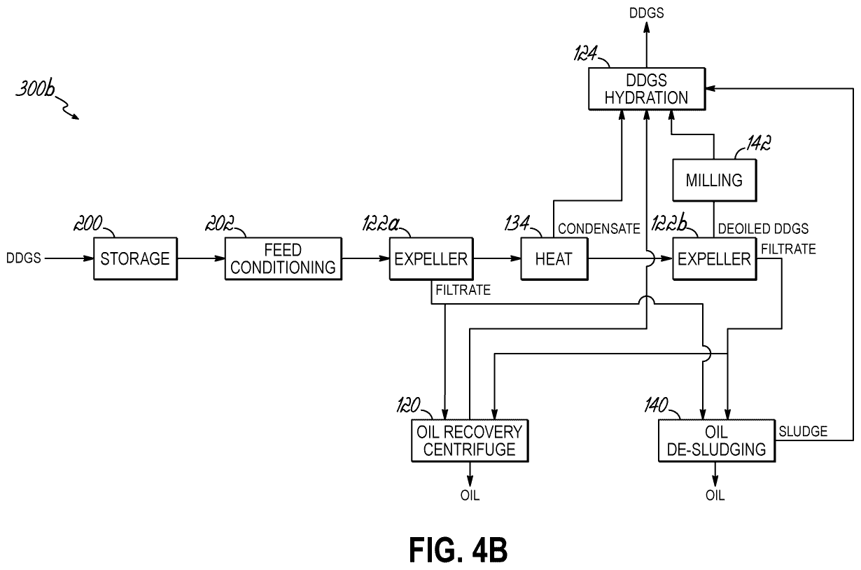

In accordance with the present invention, FIG. 4 shows another embodiment of a method and system for recovering oil in a dry grind process, collectively numeral 300, like the method and system just described in FIG. 3 but with variations thereto. To that end, certain reference numerals used in FIG. 3 are used here to represent like devices and/or steps in the method and system 300. The variations of the embodiment of FIG. 4 are discussed hereinbelow.

As shown in FIG. 4, the optional second dryer 132 of FIG. 3 has been removed such that that the dried fiber cake from the first dryer 130 can be sent and subjected directly to the first oil press device 122a, e.g., the first expeller press, to extract oil from the dried fiber cake by pressing the fiber cake to recover oil trapped within the fiber cake.

The moisture content of the pressed fiber cake after the first oil press device 122a can be from about 2% to 9%. In another example, the moisture content of the pressed fiber cake can be from about 1% to 5% or from about 2% to 4%. In another example, the moisture content of the pressed fiber cake can be about 2%. In another example, the moisture content of the pressed fiber cake can be from about 5% or less. In another example, the moisture content of the pressed fiber cake can be from about 4.5% or less. In another example, the moisture content of the pressed fiber cake can be from about 4% or less. The temperature of the first oil press device 122a can be from about 175° F. to about 250° F. In another example, the temperature can be from about 205° F. to about 225° F. In another example, the temperature can be from about 205° F. to about 215° F. In another example, the temperature can be about 210° F. In one example, the pressure can be from 1 psi to about 6,000 psi. In another example, the pressure can be from about 3,000 psi to about 5,000 psi. In another example, the pressure can be from about 1,000 psi to about 3,000 psi. In another example, the pressure can be from about 2,000 psi to about 4,000 psi. In still another example, the pressure can be greater than 6,000 psi. These are exemplary examples of moisture, pressure, and temperature and one skilled in the art can determine the optimal conditions as needed for oil recovery.

The pressed cake from the first oil press 122a then can be subjected to the optional and additional heating step(s) 134 whereat the further dried fiber cake can be subjected to the heated device, such as the heated screw conveyer (e.g., a steam jacketed screw conveyer), or the like, and/or the third dryer, such as the trim dryer or the like, at a desired temperature(s) to remove any additional or desired moisture from the fiber cake in the form of water vapor/condensate so as to provide a desired moisture level in the further dried fiber cake. Condensate from the additional heating step(s) 134 can be combined with the combined soluble solids portion and deoiled insoluble solids portion, along with the sludge, at step 124 to further hydrate/rehydrate the combination to provide a distillers dry grains with solubles (DDGS), which may be sold to dairy and beef feedlots, as discussed above.

The moisture content of the further dried fiber cake after the optional and additional heating step(s) 134, for example, can be from about 2% to 9%. In another example, the moisture content of the dried fiber cake can be from about 1% to 5% or from about 2% to 4%. In another example, the moisture content of the dried fiber cake can be about 2%. In another example, the moisture content of the dried fiber cake can be from about 5% or less. In another example, the moisture content of the dried fiber cake can be from about 4.5% or less. In another example, the moisture content of the dried fiber cake can be from about 4% or less. The temperature of the heated device(s), such as the screw conveyor and/or third dryer, at step 134 can be from about 175° F. to about 250° F. In another example, the temperature can be from about 205° F. to about 225° F. In another example, the temperature can be from about 205° F. to about 215° F. In another example, the temperature can be about 210° F. The temperature of the heated devices of the optional and additional heating step(s) 134 may be the same as, lower, or higher than the temperature of one another, the first dryer 130, or the first oil press device 122a, as desired. These are exemplary examples of moisture and temperature and one skilled in the art can determine the optimal conditions as needed for oil recovery.

Then, the pressed and further dried cake from the additional heating step(s) 134 can be sent to the second oil press 122b, e.g., the second expeller press, to again extract additional oil from the pressed and dried fiber cake by further pressing the fiber cake to recover additional oil trapped within the fiber cake. As indicated above, it is contemplated that the expeller press can be replaced by a standard screw press. All or a portion of the further pressed fiber cake or deoiled insoluble solids (which may or may not include soluble solids, as indicated above) from the second oil press 122b device can be combined with soluble solids and further subjected to hydration step 124 including initially being subjected to milling step 142 prior to the hydration step 124, as discussed above.

In one example, the moisture content of the pressed fiber cake after the second oil press device 122b can be from about 2% to 9%. In another example, the moisture content of the pressed fiber cake can be from about 1% to 5% or from about 2% to 4%. In another example, the moisture content of the pressed fiber cake can be about 2%. In another example, the moisture content of the pressed fiber cake can be from about 5% or less. In another example, the moisture content of the pressed fiber cake can be from about 4.5% or less. In another example, the moisture content of the pressed fiber cake can be from about 4% or less. The temperature of the second oil press device 122b can be from about 175° F. to about 250° F. In another example, the temperature can be from about 205° F. to about 225° F. In another example, the temperature can be from about 205° F. to about 215° F. In another example, the temperature can be about 210° F. The temperature of the first and second oil press devices 122a, 122b may be the same as, lower, or higher than the temperature of one another, the first dryer 130, or the heated device of the additional heating steps 134, as desired. In one example, the pressure used in the second oil press device 122b can be from 1 psi to about 6,000 psi. In another example, the pressure can be from about 3,000 psi to about 5,000 psi. In another example, the pressure can be from about 1,000 psi to about 3,000 psi. In another example, the pressure can be from about 2,000 psi to about 4,000 psi. In still another example, the pressure can be greater than 6,000 psi. These are exemplary examples of moisture, pressure, and temperature and one skilled in the art can determine the optimal conditions as needed for oil recovery.

In an alternate embodiment, it is contemplated that the first and/or second oil press devices 122a, 122b can be replaced by a standard screw press.

As further shown in FIG. 4, the oil filtrate from the first oil press device 122a can be sent to oil recovery centrifuge 120, such as by being combined with the water soluble solid portion prior to and/or after the evaporators 116a-c, and/or sent to the hydration step 124 to be combined with the deoiled insoluble solids and soluble solids, along with the sludge. The oil filtrate from the second oil press device 122b can be sent to the oil desludging step 140 whereat the oil filtrate can be subjected to the second oil recovery centrifuge so that oil can be removed therefrom. The resulting sludge may be combined with the deoiled DDG(S) and soluble solids, which can be further hydrated at step 124, as discussed above. Alternatively, all or a portion of the oil filtrate from the second oil press 122b, like the oil filtrate from the first oil press 122a, can be sent to oil recovery centrifuge 120, such as by being rejoined with the water soluble solids portion just after the evaporators 116a-c, as shown in FIG. 4.

Also, in FIG. 4, the optional second set of evaporators 116d-e has been removed such that at least a portion of the soluble solids from the oil recovery centrifuge 120 may be combined directly with the deoiled insoluble solids portion received from the second oil press device 122b to provide a distillers dry grains with solubles (DDGS), which may be further hydrated and/or sold to dairy and beef feedlots. In addition, as shown in FIG. 4, at least a portion of the soluble solids stream from the oil recovery centrifuge 120 can be combined with the insoluble solids stream just downstream of the centrifuge 104, such as after the first dryer 130, as discussed above.

In accordance with the present invention, FIG. 4A shows a variation of the method and system of FIG. 4 for recovering oil in a dry grind biochemical process. Here, in this method and system for recovering oil in a dry grind process, collectively numeral 300a, the optional paddle screen 102 and the optional pressure screen 106 in the method 300 of FIG. 4 have been removed. In addition, the nozzle centrifuge 110 of FIG. 3, likewise, is considered optional here and has been removed such that the centrate (solubles)/thin stillage from the centrifuge 104 can be sent directly to evaporators 116a-c, and not the water soluble solids portion. In one example and as discussed above with respect to FIG. 4, the centrifuge 104 can be replaced with a decanter centrifuge, such as a solid bowl decanter. Notably, in view of the removal of the nozzle centrifuge 110, the protein portion is not separated out of the centrate and a high protein corn meal is not produced in this method 300a. Instead, the protein remains with the centrate, which is subjected to evaporation at evaporators 116a-c and further processed in accordance with the detailed discussion of the method 300 of FIG. 4 to increase oil yield/recovery yet provide for a DDGS that includes a higher protein content relative to the DDGS of FIG. 4, which may be sold to beef and dairy feedlots as cattle feed or other animal feed markets, such as fish feed or the pet food market, for example.

In accordance with the present invention, FIG. 4B shows another variation of the method and system of FIG. 4 for recovering oil in a dry grind biochemical process. Here, in this method and system for recovering oil a dry grind biochemical process, collectively numeral 300b, it has been adapted, similar to FIG. 3B, to receive DDGS, for example, that has been produced via a dry grind biochemical process at a different source/location, e.g., an alcohol plant, which could employ a method and system that is the same as or similar to that shown and described in FIG. 1, 4, or 4A. The received DDGS initially may be stored at storage step 200 and optionally conditioned at feed conditioning step 202 before being subjected to further treatment at first expeller/first oil press device 122a, as described above in FIG. 4, to recover oil still contained therein. At optional feed conditioning step 202, the DDGS can be subjected to a mixing device, such as a paddle mixer or the like, to break up DDGS chunks, for example, and help provide a generally uniform particle size therein. Liquid, e.g., water, can be added to the mixing device, as needed/desired, to adjust the moisture content of the DDGS. The temperature of the DDGS may be adjusted, as needed/desired, such as by heating the same before, at, and or after the mixing device, which can also assist in moisture control of the DDGS. In addition, the DDGS can be treated with a processing aid such as a binding agent, e.g., starch, steam, syrup, and the like, that can be added to the DDGS, such as before, at, and/or after the mixing device, to allow for fines (such as fine fiber) to be bound with the larger pericarp fiber particles and pass through the oil press device 122, allowing for greater oil recovery and less downstream processing needs to remove the fines at the oil de-sludging step 140. Other processing aids alternately or additionally may be used as well including, for example, enzymes, such as phytase, protease, cellulase, hemicellulase, xylanase, beta-glucanase, and the like, flocculants, surfactants, demulsifiers, pH adjusters, such as sulfuric acid, hydrochloric acid, sodium hydroxide, and the like, which can further assist with oil recovery.

With continuing reference to FIG. 4B, the DDGS, which may be optionally conditioned, now can be subjected directly to first oil press device 122a and further processed in accordance with the detailed discussion of the method 300 of FIG. 4 to increase oil yield/recovery. And, as shown, all other steps/machinery for processing whole stillage have been removed. As such, with this standalone DDGS/oil recovery method and system 300b, there is no high protein grain (corn) meal produced with this specific setup, as with the method and system 300 of FIG. 4. In addition, it is noted that while both the oil recovery centrifuge 120 and the oil desludging step 140 are shown in FIG. 4B, only one of these may be present/needed. That is, all of the oil filtrate from both the first oil press device 122a and the second oil press device 122b can be sent to the oil recovery centrifuge 120 or to the oil de-sludging step 140. And, if used, there is no soluble solids stream being sent from the oil recovery centrifuge 120 to the hydration step 124, for example, but rather the remaining filtrate from one or both of the oil filtrate streams is sent, which would be akin to the sludge from the oil de-sludging step 140. Also, while DDGS is shown here as having been received from another location, it should be understood that MDGS or DWGS may be contemplated as options for use in this method and system 300b as well.

While the present invention has been illustrated by a description of various embodiments and while these embodiments have been described in considerable detail, it is not the intention of the applicant to restrict or in any way limit the scope of the appended claims to such detail. For example, while the DDGS can be treated, as discussed, with various processing aids such as before, at, and/or after the mixing device in FIGS. 2B, 3B, and 4B, the DDG or DWG, with or without solubles, similarly can be treated with one or more of the various processing aids such as before, at, and/or after the mixing device in any of FIGS. 2, 2A, 3, 3A, 4, or 4A, such as to allow for fines (such as fine fiber) to be bound with the larger pericarp fiber particles and pass through the oil press device(s) 122a, 122b, allowing for greater oil recovery and less downstream processing needs to remove the fines at the oil de-sludging step 140. Said processing aids may be, for example, binding agents, such as starch, steam, syrup, and the like, enzymes, such as phytase, protease, cellulase, hemicellulase, xylanase, beta-glucanase, and the like, flocculants, surfactants, demulsifiers, pH adjusters, such as sulfuric acid, hydrochloric acid, sodium hydroxide, and the like, which can further assist with oil recovery. Additional advantages and modifications will readily appear to those skilled in the art. Thus, the invention in its broader aspects is therefore not limited to the specific details, representative apparatus and method and illustrative example shown and described. Accordingly, departures may be made from such details without departing from the spirit or scope of applicant's general inventive concept.

1. A method for recovering oil from an insoluble solids portion comprising;
separating a whole stillage byproduct into an insoluble solids portion, including fiber and germ with bound oil, and a solubles portion, including soluble solids;
drying the insoluble solids portion to provide a dried insoluble solids portion, including the fiber and germ;
pressing the dried insoluble solids portion to free bound oil from the germ and to provide a first filtrate, including the free oil, and a first deoiled insoluble solids portion, including pressed fiber and germ; and
recovering oil from the first filtrate to provide an oil co-product.

2. The method of claim 1 wherein pressing the dried insoluble solids portion comprises pressing the dried insoluble solids portion via a heated oil press to free bound oil from the germ and to provide the first filtrate, including the free oil, and the first deoiled insoluble solids portion, including pressed fiber and germ.

3. The method of claim 1 wherein pressing the dried insoluble solids portion comprises pressing the dried insoluble solids portion via a screw press to free bound oil from the germ and to provide the first filtrate, including the free oil, and the first deoiled insoluble solids portion, including pressed fiber and germ.

4. The method of claim 1 wherein drying the insoluble solids portion includes drying via a first dryer to provide the dried insoluble solids portion, including the fiber and germ.

5. The method of claim 4 wherein a moisture content of the dried insoluble solids portion after the first dryer can be about 30% or more.

6. The method of claim 4 wherein a temperature of the first dryer can be from about 175° F. to about 250° F.

7. The method of claim 1 further comprising drying the dried insoluble solids portion via a second dryer.

8. The method of claim 7 wherein a moisture content of the dried insoluble solids portion after the second dryer is from about 2% to 12%.

9. The method of claim 7 further comprising heating the further dried insoluble solids portion after the second dryer so that a moisture content of the heated insoluble solids portion is from about 2% to 9%.

10. The method of claim 1 further comprising separating the solubles portion into a protein portion, including protein, and a water soluble solids portion, including the soluble solids.

11. The method of claim 1 further comprising combining at least a portion of the soluble solids with at least a portion of the dried insoluble solids after drying the insoluble solids portion.

12. The method of claim 1 wherein recovering oil from the filtrate to provide an oil co-product comprises recovering oil via an oil recovery centrifuge from the filtrate to provide the oil co-product.

13. The method of claim 1 further comprising milling at least a portion of the deoiled insoluble solids portion.

14. The method of claim 1 further comprising separating soluble solids from the water soluble solids portion to provide a soluble solids portion, and further combining at least a portion of the soluble solids with the deoiled insoluble solids portion.

15. The method of claim 1 further comprising combining the deoiled insoluble solids portion with vapor released from drying the insoluble solids portion.

16. The method of claim 1 further comprising pressing the first deoiled insoluble solids portion, including pressed fiber and germ, to free additional bound oil from the germ and to provide a second filtrate, including the free oil, and a second deoiled insoluble solids portion, including pressed fiber and germ, and wherein recovering oil from the first filtrate to provide an oil co-product comprises recovering oil from the first and/or second filtrate to provide an oil co-product.

17. The method of claim 1 further comprising heating the first deoiled insoluble solids portion followed by pressing the heated first deoiled insoluble solids portion, including pressed fiber and germ, to free additional bound oil from the germ and to provide a second filtrate, including the free oil, and a second deoiled insoluble solids portion, including pressed fiber and germ, and wherein recovering oil from the first filtrate to provide an oil co-product comprises recovering oil from the first and/or second filtrate to provide an oil co-product.

18. A method for recovering oil from an insoluble solids portion comprising;
conditioning distillers grains with or without solubles that defines an insoluble solids portion and includes fiber and germ with bound oil by mixing and/or rehydrating the distillers grains with or without solubles;
pressing the conditioned distillers grains with or without solubles to free bound oil from the germ and to provide a first filtrate, including the free oil, and a first deoiled distillers grains with or without solubles, including pressed fiber and germ; and
recovering oil from the first filtrate to provide an oil co-product.

19. The method of claim 18 further comprising heating the conditioned distillers grains with or without solubles and pressing the heated distillers grains with or without solubles to free bound oil from the germ and to provide the first filtrate, including the free oil, and the first deoiled distillers grains with or without solubles, including pressed fiber and germ.

20. The method of claim 18 further comprising pressing the first deoiled distillers grains with or without solubles, including pressed fiber and germ, to free additional bound oil from the germ and to provide a second filtrate, including the free oil, and a second distillers grains with or without solubles, including pressed fiber and germ, and wherein recovering oil from the first filtrate to provide an oil co-product comprises recovering oil from the first and/or second filtrate to provide an oil co-product.

21. A system for recovering oil from an insoluble solids portion comprising:
a first apparatus that receives the whole stillage byproduct and is configured to separate the whole stillage byproduct into an insoluble solids portion, including fiber and germ with bound oil, and a solubles portion, including soluble solids;
a first drying device that is situated after the first apparatus and that receives the insoluble solids portion, the first drying device dries the insoluble solids portion to provide a dried insoluble solids portion, including the fiber and germ;
a second apparatus that is situated after the first drying device and that receives the dried insoluble solids portion, the second apparatus configured to press the dried insoluble solids portion to free bound oil from the germ and provide a first filtrate, including the free oil, and a first deoiled insoluble solids portion, including pressed fiber and germ; and
an oil recovery device that is situated after the second apparatus and that receives the first filtrate, the oil recovery device configured to recover oil from the first filtrate to provide an oil co-product.

22. The system of claim 21 wherein the first drying device is selected from a dryer.

23. The system of claim 21 wherein the second apparatus is selected from a heated oil press or a screw press.

24. The system of claim 21 wherein the oil recovery device is an oil recovery centrifuge.

25. The system of claim 21 further comprising a second drying device that is situated after the first drying device and that receives the dried insoluble solids portion, the second drying device dries the dried insoluble solids portion to provide a further dried insoluble solids portion, including the fiber and germ.

26. The system of claim 25 further comprising a heating device that is situated after the second drying device and that receives the further dried insoluble solids portion, the heating device configured to heat the further dried insoluble solids portion.

27. The system of claim 21 further comprising a third apparatus that is situated after the second apparatus and that receives the first deoiled insoluble solids portion, the third apparatus configured to press the first deoiled insoluble solids portion, including pressed fiber and germ, to free additional bound oil from the germ and to provide a second filtrate, including the free oil, and a second deoiled insoluble solids portion, including pressed fiber and germ, and wherein the oil recovery device is situated after the third apparatus and receives the first and/or second filtrate, the oil recovery device configured to recover oil from the first and/or second filtrate to provide an oil co-product.

28. The system of claim 21 further comprising:
a heating device that is situated after the second apparatus and that receives the first deoiled insoluble solids portion, the heating device configured to heat the first deoiled insoluble solids portion,
a third apparatus that is situated after the heating device and that receives the heated first deoiled insoluble solids portion, the third apparatus configured to press the heated first deoiled insoluble solids portion, including pressed fiber and germ, to free additional bound oil from the germ and to provide a second filtrate, including the free oil, and a second deoiled insoluble solids portion, including pressed fiber and germ, and
wherein the oil recovery device is situated after the third apparatus and receives the first and/or second filtrate, the oil recovery device configured to recover oil from the first and/or second filtrate to provide an oil co-product.
